(19) United States
(12) Patent Application Publication
(10) Pub. No.: US 2024/0060919 A1
(43) Pub. Date: Feb. 22, 2024

(54) CHEMICAL REACTION HAZARD ANALYSIS METHOD

(71) Applicant: Shenyang Research Institute of Chemical Industry Ltd., Shenyang (CN)

(72) Inventors: ChunSheng Cheng, Shenyang (CN); Zhenyun Wei, Shenyang (CN); QuanGuo Li, Shenyang (CN); Xiaohua Ma, Shenyang (CN); Peng Li, Shenyang (CN); Xuan Liu, Shenyang (CN); Rong Kong, Shenyang (CN); Chuang Zhao, Shenyang (CN)

(21) Appl. No.: 18/450,418

(22) Filed: Aug. 16, 2023

(30) Foreign Application Priority Data
Aug. 17, 2022 (CN) 2022109852730

(51) Int. Cl. G01N 25/48; G01N 31/12; B01J 19/00

(52) U.S. Cl. CPC G01N 25/48; G01N 31/12; B01J 19/002; B01J 2219/00243; B01J 2219/00261; B01J 2219/00051; B01J 2219/00162

(57) ABSTRACT

A chemical reaction hazard analysis method is disclosed. Safety data and preventive measures of a chemical reaction are obtained through analysis of material stability, reaction process hazards and reaction runaway. The method can shorten a distance from laboratory to industrialization and realize an organic combination and application of technology, safety and engineer. The data obtain by the method can provide underlying basic data for process design, engineer amplification and the like, and lay a foundation for realizing process safety, improving quality and increasing efficiency.

### TECHNICAL FIELD

The disclosure relates to the technical field of chemical reaction hazards, and particularly to a chemical reaction hazard analysis method.

### BACKGROUND

The chemical industry uses chemicals and performs various chemical reactions, involving various disciplinary fields such as transformation and transmission of substances, energy, engineering and information. For a long time, the lack of systematic chemical safety technology system around scientific issues of the transformation and transmission of energy has led to the lack of underlying safety data and the lack of safety technology and data support for process safety, process designs and risk management and risk control. Therefore, it is urgent to develop a hazard analysis method for chemical reaction process to meet needs of the industry and improve the level of chemical safety management and safety production.

A literature of chemical reaction hazards and risk control of fine chemical industry discloses methods of reaction safety risk assessment, including chemical reaction hazard identification and reaction safety risk assessment, and this literature introduces methods of event tree analysis and decomposition heat assessment. Chinese patent with publication No. CN113470757A discloses a research method of thermal hazard analysis method of diazotization process, describes the thermal hazard analysis method and data acquisition of diazotization reaction, and applies the obtained data to safety risk assessment. Chinese patent with publication No. CN113724794A discloses a method for detecting reaction runaway, and describes a strategy for detecting and controlling reaction runaway. Chinese patent with publication No. CN109949874A discloses a risk degree grading method for fine chemical production process safety evaluation, and the evaluation method is formed by forming a risk index. In the above-mentioned literature and patents, the research of reaction hazards in terms of chemicals, chemical reactions and reaction runaway fails to form a system for data acquisition and construct a correlation between the data.

### SUMMARY

The objective of the disclosure is to provide a chemical reaction hazard analysis method.

To achieve the above objective, technical solutions adopted by the disclosure are as follows:

A chemical reaction hazard analysis method is disclosed, and safety data and preventive measures for a chemical reaction are obtained by analyzing material stability, reaction process risks, and reaction runaway; specifically, the chemical reaction hazard analysis method includes the following steps:
(1) thermal stability analysis of chemicals:
comparing self-state parameters of the chemicals in the chemical reaction with state parameters of the chemicals produced in the chemical reaction by a joint method of differential heat-pressure heat-insulation heat, determining a risk critical value according to a degree of danger, and obtaining a safe critical value of the chemicals for operation, use, storage and transportation;
(2) reaction process risk analysis:
determining process designs and interlock control parameters of industrialized chemical reaction process according to apparent thermodynamic parameters and apparent kinetic parameters of the chemical reaction;
(3) reaction runaway analysis:
analyzing reaction conditions of each step in the chemical reaction and runaway situations of a reaction device by adiabatic accelerating rate calorimetry, low temperature inert adiabatic calorimetry and phi 1 calorimetry, and obtaining a risk control critical value in the runaway situations by testing; and determining a chemical reaction hazard according to the risk control critical value, and formulating a risk control measure correspondingly.

In an embodiment, the risk control measure includes a target addition amount of each chemical; and the chemical reaction hazard analysis method further includes: adding the chemical of the target addition amount once into a reaction kettle for the chemical reaction.

In an embodiment, the thermal stability analysis of the chemicals in the step (1), includes: performing tests of differential heat, pressure heat and insulation heat on samples with different quality grades of the chemicals, comparing data obtained through the tests with the state parameters of the samples produced in the chemical reaction, and determining the risk critical value according to the degree of danger, that is, obtaining the safe critical value of the chemicals for the operation, the use, the storage and the transportation.

In an embodiment, the chemicals include: one or more of raw materials, intermediates, finished products and wastes involved in the chemical reaction.

In an embodiment, in the step (1), the joint method of differential heat-pressure heat-insulation heat includes: differential scanning calorimetry, pressure screening calorimetry and adiabatic accelerating rate calorimetry.

In an embodiment, the self-state parameters (i.e., characteristic parameters) of the chemicals and the state parameters produced in the chemical reaction include an initial decomposition temperature, a heat release during decomposition, a temperature rise rate during decomposition, a pressure rise rate and a time to maximum rate (TMR_{ad}).

In an embodiment, the self-state parameters of the chemicals and the state parameters produced in the chemical reaction are compared, such as the initial decomposition temperature is compared with an operating temperature of a material to make it clear that the operating temperature of the material meets the safety requirement; a degree of risk of the material during decomposition is analyzed based on the heat release during decomposition, the temperature rise rate during decomposition and the pressure rise rate, and the corresponding risk control measure is formulated according to the degree of risk; the time to maximum rate (TMR_{ad}) is obtained according to the decomposition activation energy and a relationship (TMR_{ad} curve) between the time to maximum rate and temperature is obtained, and thus requirements for safe operation time of the material at different temperatures are determined.

In an embodiment, the step (2) includes: obtaining the apparent thermodynamic parameters and the apparent kinetic parameters of the chemical reaction by reaction calorimetry, microcalorimetry, pressure calorimetry, differential calorimetry and adiabatic calorimetry; the apparent thermodynamic parameters include an apparent reaction heat, an exothermic rate, an adiabatic temperature rise, a heat accumulation and a heat conversion rate; and the apparent kinetic parameters include a reaction kinetic equation, an activation energy, a pre-exponential factor and a reaction order.

In an embodiment, the step (2) includes: performing an industrial design according to the apparent thermodynamic parameters, and determining a heat exchange area, a temperature control scheme and reaction conditions of a reactor; performing amplification condition simulation according to the apparent kinetic parameters, and determining operation conditions, the process designs and the interlock control parameters of the industrialized chemical reaction process; and the operation conditions include an industrial engineering mode, a reaction temperature, a heating and cooling time and a feeding speed.

In an embodiment, the step (2) specifically includes the following steps:
(a) apparent reaction heat test of different reaction forms, such as batch, semi-batch process and continuous flow, is performed, for example, using full-automatic reaction calorimeter, high-pressure reaction calorimeter, low-temperature reaction calorimeter, continuous flow reaction calorimeter and so on; the apparent heat data of the process is obtained and includes chemical reaction heat, solution heat, crystallization precipitation heat, mixing heat, etc., as well as the instantaneous maximum exothermic power, heat accumulation, and the maximum temperature of the synthesis reaction (MTSR) that the runaway system can reach;
(b) the second decomposition test and analysis of feed liquid of the reaction system at end of reaction are performed, and the initial decomposition temperature, the temperature corresponding to the time to maximum rate, adiabatic temperature rise and other data of the test sample are obtained; and
(c) process designs and interlock control parameters of industrial chemical reaction process are determined based on the reaction calorimetry results.

In an embodiment, the reaction conditions of each step in the chemical reaction and the runaway situations of the reaction device in the step (3) include temperature runaway, pressure runaway, feeding runaway, process condition deviation, cooling failure and stirring failure.

The advantages of the disclosure are as follows:
The disclosure performs chemical reaction hazard analysis for chemicals, chemical reactions, and reaction runaway involved in the chemical process, the method of the disclosure provides a safe path for the transformation of processes into engineering, and controls the chemical process safety, precision technology, fine design, and fine production. The method of the disclosure solves the problems of unsystematic research methods, incomplete data, and insufficient support for process development and engineering design in the prior art. The correlation relationship between data has been established through the method of the disclosure, achieving mutual calibration of methods such as differential heat, pressure heat, and insulation heat. Furthermore, the accuracy of industrial application of experimental data is improved through data fitting and dynamic analysis, the process research is performed on chemical reactions to obtain apparent thermodynamic and kinetic parameters of the reaction process, the risk of uncontrolled processes is analyzed, and basic data is provided for establishing risk control measures such as emergency termination and overpressure relief. The method of the disclosure can shorten a distance from laboratory to industrialization, and achieve the organic combination and application of technology, safety, and engineering. The data obtained by the method of the disclosure can provide underlying basic data for process design, engineering amplification, etc., laying the foundation for achieving process safety and improving quality and efficiency.

### DETAILED DESCRIPTION OF EMBODIMENTS

The specific embodiments of the disclosure will be further described with embodiments. It should be pointed out that the specific embodiments described here are only for the purpose of explaining the disclosure, and are not limited to the disclosure.

### Embodiment 1

The following reaction is taken as an example to study a chemical reaction hazard of this reaction, specifically:

1. Thermal Stability Analysis of Chemicals

In order to investigate the thermal stability of a sample, a joint testing method is used to perform thermal stability test and analysis on boscalid to obtain the thermal stability information of the sample.

1.1 The boscalid of 2.1000 milligrams (mg) is taken and performed milligram-level thermal stability test using a method of differential scanning calorimetry (DSC). The sample undergoes exothermic decomposition at 248.3° C., and within a testing range of 248.3-327.9° C., an exothermic quantity (i.e., heat release) of the sample is 281.8 Joules per gram (J·g⁻¹) (calculated by a weight of the sample).

1.2 The boscalid of 2.2545 grams (g) is taken and performed gram-level thermal stability test using a method of rapid screening calorimetry. The sample undergoes outgassing decomposition at 235° C., and a pressure does not return after this test system is cooled, which further explains that the sample is decomposed to generate gas during the test.

1.3 The boscalid of 2.6546 g is taken and performed a gram-level adiabatic thermal stability test using a method of adiabatic accelerating rate calorimetry. The sample undergoes exothermic decomposition at 196.5° C. Within a testing range of 196.5-231.4° C., an exothermic quantity by the decomposition is 155 J·g⁻¹ (calculated by a weight of the sample), and an adiabatic temperature rise is 71.8 Kelvin (K), the maximum temperature rise rate during the decomposition process is 0.4 degree Celsius per minute (° C.·min⁻¹), and the maximum pressure rise rate at 216.7° C. is 0.1 bar·min⁻¹.

1.4 The thermal stability test results of the above mentioned boscalid with different weights are fitted to obtain a sample decomposition activation energy of 132-415 kJ·mol⁻¹. Under the adiabatic condition, a temperature T_{D2} is 200° C., a temperature T_{D4} is 195° C., a temperature T_{D8} is 185° C., a temperature T_{D24} is 170° C., and a temperature T_{D168} is 150° C. when a time to maximum reaction rate of thermal decomposition is 2 hours (assuming a Phi system is 1.05).

Under the adiabatic condition, the corresponding temperatures T_{D8} and T_{D24} are tested when the times to maximum reaction rates of sample decomposition are respectively 8 hours and 24 hours, which is very important for risk control in emergency.

The initial decomposition temperature and the time to maximum reaction rate at different temperatures of the sample in the chemical reaction can be known from the above. According to the test results, the initial decomposition temperature of boscalid is 196.5° C. and T_{D24} is 170° C. In the process of industrialization, the maximum temperature for enterprises to use boscalid is 100° C., and the operation time is 15 hours. The boscalid is stable in the range of temperature and operation time, and the possibility of risks under the process conditions is low.

2. Reaction Process Risk Analysis

The following process is taken as an example:

Water, hydrochloric acid and aniline are added into a reaction kettle, a temperature of the reaction kettle is controlled to be in a range of 0 to −20° C., then sodium nitrite aqueous solution is dropwise added into the reaction kettle, and the temperature remains unchanged until the end of the reaction after the feeding is finished.

2.1 For this reaction, the apparent reaction heat (based on a weight of the aniline) is −645.9 kJ·kg⁻¹ by reaction calorimetry test. The instantaneous maximum exothermic rate (i.e., instantaneous maximum heat release rate) during the feeding process is 56.2 W·kg⁻¹ (based on the instantaneous weight of feed liquid), and the adiabatic temperature rise is 4.9 K. At the end of feeding, the conversion rate of reaction heat is 98.8%. The upper limit temperature of the process is 0° C., and the maximum temperature of the synthesis reaction (MTSR) that the system can reach after cooling failure is 4.9° C.

According to the test results, the apparent reaction heat can be used in engineering design to determine the engineering mode, heat exchange area of the reactor, temperature control scheme and reaction conditions. The heat accumulation of the reaction is small, and it is a feeding-controlled reaction. According to the MTSR, the highest process temperature of the reaction after thermal runaway is determined, which is used to evaluate the thermal safety of the reaction system in the future.

2.2 The joint test method is used to perform secondary decomposition safety test and analysis of the feed liquid of the system at end of reaction: the joint test of differential heat-pressure heat-insulation heat is performed, and the results show that the sample is decomposed by outgassing at 50.1° C. and is decomposed exothermically at 55.1° C., and the decomposition heat is 29.5 J·g⁻¹ (based on a weight of the sample) within a test range of 55.1-60.9° C., and the maximum temperature rise rate during the decomposition is 0.1° C.·min⁻¹ and the maximum pressure rise rate during the decomposition is 0.1 bar·min⁻¹.

2.3 The test results of the feed liquid with different weights of the system at the end of reaction are fitted, and decomposition kinetics analysis is performed, it is obtained that the activation energy of sample decomposition reaction is 62.8-143.6 kJ·mol⁻¹, and a temperature T_{D2} is 56° C., a temperature T_{D4} is 50° C., a temperature T_{D8} is 42° C., a temperature T_{D24} is 32° C., and a temperature T_{D168} is 16° C. when the time to maximum reaction rate reaches 2 h under the adiabatic condition.

According to the above test and analysis, the safety of the reaction at the runaway maximum temperature MTSR is evaluated. For example, the maximum temperature of the reaction runaway is 4.9° C., the feed liquid at the end of the reaction is decomposed at 55.1° C., and the corresponding temperature is 32° C. when the time to maximum reaction rate reaches 24 h under the adiabatic condition, all of which are at a certain distance from MTSR. Under the process conditions, the thermal stability of the reaction system is good, and once cooling failure occurs, the feed is cut off immediately, and the possibility of danger is not high.

3. Risk Analysis of Reaction Runaway

The following process is taken as an example:

At room temperature, 12.0 g of crotonyl alcohol and 0.02 g of catalyst are added into a reaction kettle, a reaction temperature is controlled to be in a range of 65° C. to 85° C., and 61.7 g of silicone oil is gradually dropwise added for 6-8 hours. After the dropwise addition is completed, the reaction is kept at the temperature for 0-2 h.

3.1 Simulating data of the highest temperature and pressure reached by the reaction system when the feeding fails.

After adding 20% silicone oil once, a pressure rise rate of the reaction system reaches the maximum value of 0.32 bar·min⁻¹, a system pressure increases from 1.00 bar to 2.00 bar, a reaction heat of the reaction system is −220.3 kJ·kg⁻¹ (based on the weight of silicone oil), and the temperature rise is 49.4 K.

After adding 40% silicone oil once, the pressure rise rate of the reaction system reaches the maximum value of 5.57 bar·min⁻¹, the system pressure increases from 1.00 bar to 6.20 bar, the reaction heat of the reaction system is −219.6 kJ·kg⁻¹ (based on the weight of silicone oil), and the temperature rise is 63.3 K.

After adding 60% silicone oil once, the pressure rise rate of the reaction system reaches the maximum value of 9.77 bar·min⁻¹, the system pressure increases from 1.00 bar to 8.93 bar, the reaction heat of the reaction system is −226.8 kJ·kg⁻¹ (based on the weight of silicone oil), and the temperature rise is 73.6 K.

It can be seen from the above that with the increase of single feeding amount (i.e., addition amount), the heat release, adiabatic temperature rise and maximum pressure of thermal runaway reaction increase, and the severity of reaction runaway gradually increase. The feeding amount should be strictly controlled in the production process to avoid runaway feeding.

4 Application of Reaction Hazard Analysis Results

Process conditions: ethyl sulfide is added into a reaction kettle, and then hydrogen peroxide is added dropwise into the reaction kettle. At a temperature of 35° C. to 45° C., 70% hydrogen peroxide is added within 1-2 hours in a first stage, and 30% hydrogen peroxide is added within 0.5-1 hour in a second stage. After the dropwise addition, the temperature is raised to 85-95° C. and the temperature is kept for 1.5-2.5 h until the reaction is completed.

The reaction thermodynamic test results are obtained by reaction calorimetry test. The apparent reaction heat of the reaction is 3000.0 kJ·kg⁻¹ (based on the weight of ethyl sulfide), the adiabatic temperature rise of the reaction is 360.0 K, the maximum heat accumulation during the feeding process is 9.0%, and the product content is 84.5%.

The risk of thermal runaway of this reaction is high, and there are the following problems:
(1) The instantaneous exothermic power of the reaction is high, reaching 400 W·kg⁻¹, and it is difficult to enlarge industrialization and transfer heat;
(2) There is heat accumulation in the feeding process, and there is a potential risk of thermal runaway;
(3) Solid products are precipitated during the feeding process, and a large amount of heat is released during the precipitation process, with high instantaneous heat release rate and poor controllability;
(4) According to guidelines for chemical reaction safety and risk assessment of fine chemical industry (trial), the hazard degree (i.e., risk degree) of this process is Grade 3.

Through the risk analysis results, the original process is optimized, the dropping acceleration rate is changed, and the heat accumulation is reduced. The optimized process conditions are used to verify the reaction calorimetry test. The optimized test results are as follows: the apparent reaction heat is 2700.0 kJ·kg⁻¹ (based on the weight of ethyl sulfide), the adiabatic reaction temperature rise is 315.0 K, the maximum heat accumulation during feeding is 0.1%, and the product content is 90.2%.

The optimized process is improved in the following aspects:
(1) The instantaneous exothermic power of the reaction is reduced to below 100 W·kg⁻¹, which meets demands of industrialization amplification;
(2) There is no heat accumulation in the feeding process;
(3) No solid product is precipitated during the feeding process, and the apparent heat release and adiabatic temperature rise are reduced during the feeding process;
(4) The problem of instantaneous precipitation is avoided, and the product quality is improved from 84.5% to 89.3%;
(5) According to the guidelines for chemical reaction safety and risk assessment of fine chemical industry (trial), the reaction risk is reduced to Grade 1.

### Embodiment 2

A hydrolysis process of acetic anhydride is taken as an example, acetic anhydride and water are used as raw materials, and sulfuric acid is used as catalyst, and the hydrolysis reaction is performed at a certain temperature to generate acetic acid.

1. Thermal Stability Analysis of Chemicals

In order to investigate the thermal stability of a sample, a joint testing method is used to perform thermal stability test and analysis on acetic anhydride to obtain the thermal stability information of the sample.

1.1 The acetic anhydride of 3.4730 mg is taken and performed milligram-level thermal stability test using a method of differential scanning calorimetry. Within a test range of 300° C., there is no obvious endothermic or exothermic signals of the sample.

1.2 The acetic anhydride of 3.5040 g is taken and performed gram-level thermal stability test using a method of rapid screening calorimetry. Within the test range of 300° C., there is no obvious endothermic or exothermic signals of the sample.

1.3 The acetic anhydride of 3.5170 g is taken and performed gram-level adiabatic thermal stability test using a method of adiabatic accelerating rate calorimetry. Within the test range of 300° C., there is no obvious exothermic signals in the sample.

2. Reaction Process Hazard Analysis

The following process is taken as an example:

Water of 500 g and sulfuric acid of 1 g are added into a reaction kettle, a temperature of the reaction kettle is controlled to be in a range of 40° C. to 50° C., then the acetic anhydride of 100 g is dropwise added in to the reaction kettle for 50 min, and the temperature remains unchanged until the end of the reaction after the feeding is finished.

2.1 For this reaction, the apparent reaction heat is −592.6 kJ·kg⁻¹ (based on the weight of acetic anhydride) by reaction calorimetry test. The instantaneous maximum exothermic rate during the feeding process is 42.1 W·kg⁻¹ (based on the instantaneous weight of feed liquid), the adiabatic temperature rise is 29.4 K, and the conversion rate of reaction heat is 90.1% at the end of feeding. The upper limit temperature of the process is 55° C., and the highest temperature MTSR that the system can reach after cooling failure is 84.4° C.

According to the test results, the apparent reaction heat can be used in engineering design to determine the engineering mode, heat exchange area of the reactor, temperature control scheme and reaction conditions. The reaction has a small heat accumulation during the feeding process and is a kinetic control reaction, the highest process temperature of the reaction after thermal runaway is determined according to the MTSR, which is used to evaluate the thermal safety of the reaction system in the future.

2.2 The joint test method is used to perform secondary decomposition safety test and analysis of the feed liquid of the system at the end of reaction: the joint test of differential heat-pressure heat-insulation heat is performed, and no obvious exothermic signals are generated in the test range of 300° C.

According to the above test and analysis, the safety of the reaction at the runaway maximum temperature MTSR is evaluated. For example, the runaway maximum temperature of the reaction is 84.4° C., and within the test range of 300° C., there is no obvious exothermic signals in the feed liquid of the system at the end of reaction. Under the process conditions, the thermal stability of the reaction system is good, and once cooling failure occurs, the feed is cut off immediately, and the possibility of danger is not high.

3. Hazard Analysis of Reaction Runaway

The following process is taken as an example:

At a temperature of 40-50° C., a certain weight of water and concentrated sulfuric acid are added into a reaction kettle, and a certain weight of acetic anhydride is added at once.

3.1 Simulating data of the highest temperature and pressure reached by the reaction system when the feeding fails.

After adding 20% acetic anhydride once, a temperature rise rate of the reaction system reaches the maximum value of 19.9° C.·min⁻¹, the apparent reaction heat of the reaction system is −599.5 kJ·kg⁻¹ (based on the weight of acetic anhydride), and the temperature rise is 6.9 K.

After adding 40% acetic anhydride once, the temperature rise rate of the reaction system reaches the maximum value of 39.7° C.·min⁻¹, the apparent reaction heat of the reaction system was −596.5 kJ·kg⁻¹ (based on the weight of acetic anhydride), and the temperature rise is 13.2 K.

After adding 60% acetic anhydride once, the temperature rise rate of the reaction system reaches the maximum value of 60.7° C.·min⁻¹, and the apparent reaction heat of the reaction system is −607.3 kJ·kg⁻¹ (based on the weight of acetic anhydride), and the temperature rise is 19.4 K.

It can be seen from the above that with the increase of single feeding amount, the heat release, adiabatic temperature rise and maximum pressure of thermal runaway reaction increase, and the severity of reaction runaway gradually increase. The feeding amount should be strictly controlled in the production process to avoid runaway feeding.

What is claimed is:

1. A chemical reaction hazard analysis method, comprising: obtaining safety data and preventive measures of a chemical reaction by analyzing material stability, reaction process risks and reaction runaway, specifically comprising:
step (1) thermal stability analysis of chemicals:
comparing self-state parameters of the chemicals in the chemical reaction with state parameters produced in the chemical reaction by a joint method of differential heat-pressure heat-insulation heat, determining a risk critical value according to a degree of danger, and obtaining a safe critical value of the chemicals for operation, use, storage and transportation;
step (2) reaction process risk analysis:
determining process designs and interlock control parameters of industrialized chemical reaction process according to apparent thermodynamic parameters and apparent kinetic parameters of the chemical reaction;
step (3) reaction runaway analysis:
analyzing reaction conditions of each step in the chemical reaction and runaway situations of a reaction device by adiabatic accelerating rate calorimetry, low temperature inert adiabatic calorimetry and phi 1 calorimetry, and obtaining a risk control critical value in the runaway situations by testing; and determining a chemical reaction hazard according to the risk control critical value, and formulating a risk control measure correspondingly;
wherein in the step (1), the joint method of differential heat-pressure heat-insulation heat comprises: differential scanning calorimetry, pressure screening calorimetry and adiabatic accelerating rate calorimetry;
wherein the step (2) comprises: performing an industrial design according to the apparent thermodynamic parameters, and determining a heat exchange area, a temperature control scheme and reaction conditions of a reactor; performing amplification condition simulation according to the apparent kinetic parameters, and determining operation conditions, the process designs and the interlock control parameters of the industrialized chemical reaction process; and the operation conditions comprise an industrial engineering mode, a reaction temperature, a heating and cooling time and a feeding speed.

2. The chemical reaction hazard analysis method as claimed in claim 1, wherein the thermal stability analysis of the chemicals in the step (1), comprises: performing tests of differential heat, pressure heat and insulation heat on samples with different quality grades of the chemicals, comparing data obtained through the tests with the state parameters produced in the chemical reaction, and determining the risk critical value according to the degree of danger, that is, obtaining the safe critical value of the chemicals for the operation, the use, the storage and the transportation.

3. The chemical reaction hazard analysis method as claimed in claim 2, wherein the chemicals comprise: one or more of raw materials, intermediates, finished products and wastes involved in the chemical reaction.

4. The chemical reaction hazard analysis method as claimed in claim 1, wherein the self-state parameters of the chemicals and the state parameters produced in the chemical reaction comprise an initial decomposition temperature, a heat release during decomposition, a temperature rise rate during decomposition, a pressure rise rate and a time to maximum rate.

5. The chemical reaction hazard analysis method as claimed in claim 1, wherein the step (2) comprises: obtaining the apparent thermodynamic parameters and the apparent kinetic parameters of the chemical reaction by reaction calorimetry, microcalorimetry, pressure calorimetry, differential calorimetry and adiabatic calorimetry; wherein the apparent thermodynamic parameters comprise an apparent reaction heat, an exothermic rate, an adiabatic temperature rise, a heat accumulation and a heat conversion rate; and the apparent kinetic parameters comprise a reaction kinetic equation, an activation energy, a pre-exponential factor and a reaction order.

6. The chemical reaction hazard analysis method as claimed in claim 1, wherein the reaction conditions of each step in the chemical reaction and the runaway situations of the reaction device in the step (3) comprise temperature runaway, pressure runaway, feeding runaway, process condition deviation, cooling failure and stirring failure.
